(19) United States
(12) Patent Application Publication
(10) Pub. No.: US 2026/0363559 A1
(43) Pub. Date: Nov. 19, 2026

(54) TEXTILE ELECTRODE AND CONDUCTIVE BUS COLLECTING DATA ON SKIN

(71) Applicant: Merve AYDINER, Beylikdüzü, Istanbul (TR)

(72) Inventor: Merve AYDINER, Beylikdüzü, Istanbul (TR)

(21) Appl. No.: 19/144,175

(22) PCT Filed: Mar. 14, 2023
(86) PCT No.: PCT/TR2023/050247

§ 371 Date: Jun. 27, 2025

(30) Foreign Application Priority Data
Dec. 29, 2022 (TR) 2022/021389

(51) Int. Cl. A61B 5/0205; A61B 5/00; A61B 5/01; A61B 5/11; A61B 5/242; A61B 5/27; A61B 5/28; A61B 5/291; A61B 5/296

(52) U.S. Cl. CPC A61B 5/02055; A61B 5/01; A61B 5/11; A61B 5/242; A61B 5/27; A61B 5/28; A61B 5/291; A61B 5/296; A61B 5/7267; A61B 2560/0214; A61B 2562/0204; A61B 2562/0219; A61B 2562/0247; A61B 2562/0271; A61B 2562/0285; A61B 2562/029; A61B 2562/125; A61B 2562/164; A61B 2562/227

(57) ABSTRACT

A textile-based on-skin data collecting electrode, conductive data transport pathway and IoT system integration system. This system can serve the medical, medicinal and wellness sectors where rigid electrodes are used extensively, but it is also suitable for use in clothing, sports, home textiles, automotive, space and aviation technologies, internet of things, AR/VR, mobility, smart city systems, gaming/game technologies, virtual universes and defense industry and manufacturing fields where occupational health and safety is a factor.

### TECHNICAL FIELD

The invention relates to an artificial intelligence system integrated with textile-based on-skin data collecting electrode, conductive data transport pathway and IoT system.

This invention can serve the medical, medicinal and wellness sectors where rigid electrodes are used extensively, but it is also suitable for use in clothing, sports, home textiles, automotive, space and aviation technologies, internet of things, AR/VR, mobility, smart city systems, gaming/game technologies, virtual universes and defense industry and manufacturing fields where occupational health and safety is a factor.

### BACKGROUND ART

With the development of technology, not only computers and smartphones, but also the clothes and accessories we use can turn into smart devices. The fact that user data exchange takes place with smaller devices, mounted on the user's body, provides convenience in many respects. These are called wearable technologies.

Wearable technologies are tools that can be integrated into the human body in different ways by users and are often used as various accessories. These tools, which can also be called wearable computers in the literature, have a structure that creates an almost commensalist relationship between human and computer and enriches the individual's daily life experiences. The wearable technologies include many things from sensor accessories such as smart watches, wristbands, rings and necklaces, to the Google Glass project and its derivative smart glasses, as well as smart optical lenses. Through emerging wireless network technologies, computer processors and smart sensors, most vehicles are connected to each other, search engines and people. With this Internet of Things dimension, it is obvious that wearable technologies will have an important place in human life.

As mentioned above, dressing, which is one of the basic needs of humanity such as eating, drinking and protection, has given birth to textile products that serve to dress. Since the early ages, textile products have not only been limited to clothing, but have continued to be utilized in the fields of building-construction, agriculture, agriculture, medical, automotive, composite, technical and professional fields with the evolution of technology and comfort needs, as well as in traditional products such as accessories, floor and bed covers, textile surfaces surrounding, covering and upholstering shelters. Throughout the life cycle, people actively or passively interact with textiles in all areas, from the fabric they are born into to the cloth they are buried with.

Integrating this technology into textile-based products has many advantages. One of the main features of the continuity of textile-based products is that they are suitable for 24/7 use without restricting mobility. During different daily activities, a person can adapt to the environment and activity depending on the comfort properties of the clothing. Instant data measurement is important in protecting the vital conditions of individuals, especially their health status, in emergencies, in times of need. Such an important area needs to be put into service in a way suitable for daily use. Today, smart wearable products (smart watches, smart bracelets, smart glasses, etc.) used for such operations are mostly developed under the accessories segment. One of the reasons for this is that surface electrodes in conventional use cannot be integrated into textile-based apparel in conventional textile manufacturing processes. As a result, inflexible surface electrodes are only used in secondary products such as accessories (smart watches, wristbands, etc.). Another reason is that gel electrodes can only be used in a hospital setting when the patient is stable and limit the mobility of an individual in daily activity. This type of electrodes or rigid electrodes used in measurements are detached from the user's body when the user moves, such as leans forward, so that bioelectric signals cannot be measured. Furthermore, the user is uncomfortable as these electrodes need to be fixed to the user's body with a flexible material to keep them in contact with the body. Surface electrodes limit mobility in everyday, high-activity or extreme conditions and impair the standard of comfort in clothing because they are not flexible in material. Furthermore, surface electrodes are not washable. As a result of the researches, it has been determined that the effective, continuous and regular usage life of smart wearable accessories by different user groups is 6 months. For data collection over the body, different surface electrodes need to be placed on multiple different points on the body depending on the type of data to be captured. For example, the electrodes of an electrocardiogramsor need to be placed in a specific location on the body to measure the heart rate variable, whereas the electrodes of a galvanic skin response sensor, which measures emotional sweating, are best measured with electrodes of different structures and placed in different locations on the body.

As regards to IoT, it is now an inevitable fact that the use of the internet has significantly changed our daily lives by increasing communication, information sharing and interaction between people. The new technological concept called “Internet of Things (IoT)” is defined as the intelligent connection of smart devices through objects that can sense and communicate with each other. With this technology, it is possible to monitor and collect information on almost all events in our environment (home, school, workplace, factory, city, etc.) with a large number of small-sized sensor devices that can use wireless technology.

A sensor, one of the most common concepts in wearable technologies, is a device or organ that detects certain external stimuli and responds in a distinctive way. The electrode and data transfer pathway in the invention acts as an electric charge collector or emitter in a semiconductor device. It is the element responsible for directly transferring a biopotential or skin current to the processor. In other words, this definition establishes an electrical bridge between any configured device (sensor, transducer, cable, etc.) to transmit (transmit and/or receive) a signal between the electronics (of a medical device) and the user (e.g. the user's skin, as referred to in the proposed invention). In this context, only information on wearable electrodes, textile-based electrodes and similar methods and inventions will be shared.

When the state of the art are examined, In the document no. “US20180271441A1”, the invention provides a wearable electrode, a first layer of a first material, a second material placed on the first material, the second material having a first compressive strength, the third material placed on the second material, the third material having a different second compressive strength than the second material. It includes the first compressive strength and a fourth material comprising a conductive element placed on the third material, disposed around the second material and joined to the first material. Example wearable electrodes provide data on the wearer of the active garment (e.g. wearer 1, etc.) to a local device via electrical pathways. Data may include, for example, biometric data, heart rate data, respiration. Rate data, breathing depth data, power, acceleration, speed, repetitions, burned calories data and/or taken steps data.

The application no. “US20180271441A1” TITLED “Wearable electrode and method of fabrication” is summarized as follows: A wearable electrode includes a first layer of a first material, a second material placed on the first material, the second material having a first compressive strength, the third material placed on the second material, the third material having a different second compressive strength than the second material. It includes the first compressive strength and a fourth material comprising a conductive element placed on the third material, disposed around the second material and joined to the first material. Example wearable electrodes provide data on the wearer of the active garment (e.g. wearer 1, etc.) to a local device via electrical pathways. Data may include, for example, biometric data, heart rate data, respiration.

The electrode as mentioned in the patent has four different layer structures and is composed of these multiple layers, each with different printing, conductivity, etc. The present invention aims to obtain high quality data by forming a single conductive surface with a uniform distribution.

In the document “U.S. Pat. No. 9125625B2” titled “Textile-based printable electrodes for electrochemical sensing”, the invention is summarized as follows: “Techniques and systems for the implementation of textile-based screen-printed amperometric or potentiometric sensors are described. The chemical sensor may include carbon-based electrodes for detecting at least one of NADH, hydrogen peroxide, potassium ferrocyanide, TNT or DNT in the liquid or vapor phase. In one embodiment, textile-based sensors are used to detect the presence of chemicals such as heavy metals and explosives underwater.”

The invention mentioned in the “U.S. Pat. No. 91,256,25B2” includes printing a copper and carbon-based conductive ink on a textile and adding a waterproof material to the substrate.

Our invention is performed as a result of the formation of a single conductive surface with a uniform distribution and does not require any waterproof material. The material used in the realization of the invention has waterproof, stainless properties and its strength is higher than copper.

The invention no. “EP3530183B1” titled “Wearable electrode” relates to a wearable electrode for use in obtaining a bioelectrical signal such as an electrocardiographic waveform on a daily basis.

The invention as described in patent no. “EP3530183B1” relates to a wearable electrode for obtaining electrocardiographic data.

The invention provides a textile electrode that can be adapted to wearable and dressable surfaces and is suitable for simultaneous data acquisition for multiple sensors, such as (including but not limited to) galvanic skin response, electroencephalography and electromyography, as well as electrocardiography and includes IoT integration and artificial intelligence support.

The invention “US7966052B2” Titled “textile-based Electrode” is summarized as follows: “Two garments with textile-based electrodes are described. Firstly, a wrist band for use with a system for remotely monitoring a patient with a heart condition comprises two layers of fabric with integrated textile-based electrodes. Textile-based electrodes include a fabric portion with non-conductive yarns that recover stretch and an electrically conductive field with electrically conductive yarn filaments that enable stretch recovery. The surface of the patch in contact with the skin contains a conductive field formed in the form of a continuous ring or strip. A connector connects the conductive field to a cable leading to a device. Secondly, an infant garment contains textile-based electrodes in the torso area and optionally in other areas to monitor the infant's biophysical properties while wearing the garment”.

The invention relates to a textile-based electrode or electrode system incorporated into a wrist band for remote monitoring of cardiac electrical activity and/or pacemaker function. Alternatively, such a textile-based electrode or electrode system can be incorporated into a baby garment.

The invention as described in “U.S. Pat. No. 7,966,052B2”, relates to a wearable electrode invented for obtaining electrocardiographic data, which is composed of two different layers, conductive and non-conductive.

The invention is performed by the formation of a single conductive surface with a uniform distribution, provides a textile electrode that can be adapted to wearable and dressable surfaces and is suitable for simultaneous data acquisition for multiple sensors, such as (including but not limited to) galvanic skin response, electroencephalography and electromyography, as well as electrocardiography and includes IoT integration and artificial intelligence support.

The invention no. “US20190132950A1” titled “Textile blank with seamless knitted electrode system” is summarized as follows: “A textile-based electrode system comprises a first fabric layer having an inner and an outer surface. The inner surface includes a braided electrode configured to come into contact with a user's skin. A second fabric layer is arranged and configured to contact with the outer surface of the first fabric layer. The second fabric layer comprises a braided conductive pathway configured to electrically connect to the braided electrode. Furthermore, a third layer of fabric is configured and arranged to contact with the second layer of fabric. A connector is placed on the third layer of fabric and configured to electrically connect to the braided conductive pathway. The second fabric layer can be folded around the first folding axis, and the third fabric layer can be folded around the second folding axis to place the second fabric layer in contact with the first fabric layer and the third fabric layer”.

The invention as described in patent no. “US20190132950A1” is formed by combining three different layers and involves the division of textile areas with different multiplicities.

The present invention aims to obtain high quality data by forming a single conductive surface with a uniform distribution.

The invention No. “US20170056644a1” Titled “textile-based product” is summarized as follows: “A textile article comprising a non-conductive section comprising a network of non-conductive fibers and an electrical pathway comprising a network of conductive fibers, the electrical pathway for transmitting or conducting an electrical signal when connected to a power source is provided herein. The electrical pathway and the non-conductive part are integrated into a common layer of the textile”.

In patent no. “US20170056644A1”, the present invention is the result of the configuration of three to four different conductive yarns.

The present invention aims to obtain high quality data by forming a single conductive surface with a uniform distribution.

The invention no. “US20180249767A1” titled “Biosensing garment” is summarized as follows: “The disclosed embodiments generally relate to wearable electronic bio-sensing garments. In some embodiments, an apparatus includes a biosensing garment and a plurality of electrical connectors mechanically coupled to the biosensing garment. A plurality of printed electrodes are placed on the biosensing garment and each is electrically connected to a corresponding one of a plurality of electrical connectors via a corresponding conductive pathway. The apparatus may further comprise a longitudinal member including a conductive member coupled to the plurality of elastic members in a curved pattern and configured to change from a first configuration to a second configuration as the longitudinal member flexes. The change from the first to the second configuration may result in a change in the inductance of the conductive element”.

The invention as described in the patent no. “US20180249767A1” comprises a plurality of printed electrodes in two layers, each corresponding to a different conductive pathway and having different stretch properties, which are mechanically attached to a wearable biosensing garment.

The invention is the result of the formation of a single conductive surface with a uniform distribution, allowing multiple types of data to be transmitted simultaneously to the circuit via a single conductive pathway.

The aim of the present invention no. “EP2694155B1” titled “Textile electrode device for acquisition of electrophysiological signals from the skin and manufacturing process thereof” is to provide a textile electrode device and a manufacturing process thereof that allows to perfect the known acquisition techniques of surface bioelectric signals and to fully or partially solve the previously highlighted problems. The invention as described in the patent no. “EP2694155B1” includes the steps of attaching textile fibers to an adhesive layer and placing electrodes by electrostatic flocking.

Aforementioned invention is the result of the formation of a single conductive textile surface with a uniform distribution and does not require a separate adhesive layer. The invention no. “EP1578482B1” titled “Electrode arrangement” is summarized as follows: “An electrode arrangement includes a braided electrically conductive electrode part comprising the electrically conductive yarn and at least a part of a moisture-proof electrically conductive material attached to the electrode part. During use, the electrode part and part of the material are applied to the user's skin. The moisture-proof material part is made of carbon black loaded silicone. The moisture-proof part encourages a user to sweat, and the sweat trapped between the skin and the moisture-proof part reduces skin-to-electrode contact resistance to increase the effectiveness of detecting a user's heart rate or other electrical signals generated by a user”.

The invention as described in the patent no. “EP1578482B1” comprises adding a moisture-proof black carbon-loaded silicon conductive yarn on top of a conductive yarn to promote sweating of the wearer, thereby increasing the efficiency of data collection.

The invention is the result of the formation of a single conductive surface with a uniform distribution, but the data measurement does not depend on the wearer sweating. The present invention provides high quality data analysis without being affected by humidity conditions.

To summarize in general terms, the rigid electrodes used in the formation of the aforementioned electronic interfaces cannot be used in accordance with conventional textile production methods and limit the comfort and mobility of the user. These previous invention proposals for textile-based electrode are multi-layered, mostly single-sensor compatible inventions, and they lack loT integration and the ability to support textiles with artificial intelligence.

Thus, the need to eliminate such shortcomings and disadvantages of the embodiments and practices employed in the prior art entails an improvement in the respective technical field.

### DESCRIPTION OF THE INVENTION

The present invention relates to a textile-based on-skin data collecting electrode, conductive data transport pathway, loT system integration and artificial intelligence support developed to eliminate the aforementioned disadvantages and bring new advantages to the related technical field.

The aim of the invention, which is designed by focusing on the aforementioned problems in the known state of the art, in the category of textile products, which is the only product group that people are in contact with 24/7 is to create a flexible and washable textile electrode suitable for daily use, which is developed in a way that does not restrict mobility and can be used in on-skin data analysis that requires continuous skin contact.

Any reaction of the body to the outside and any routine or acute vital activity that takes place within the body is reflected as an electrical signal. This signaling infrastructure is reflected on the skin as different conductivity values during response or activity. The invention enables these signals to be detected through the skin and transformed and transmitted into the data to be interpreted by the circuit to which it is connected. This invention can serve the medical, medicinal and wellness sectors where rigid electrodes are used extensively, but it is also suitable for use in clothing, sports, home textiles, automotive, space and aviation technologies, internet of things, AR/VR, mobility, smart city systems, gaming/game technologies, virtual universes and defense industry and manufacturing fields where occupational health and safety is a factor.

Another aim of the invention is that the conductive pathway with a multipurpose textile electrode that collects data on the skin is washable and suitable for daily wear. A conductive fabric of the invention acts as a receiver that collects and transmits data without causing discomfort to the wearer in measurements that require constant skin contact.

Another aim of the invention is to be used by everyone in daily life, as well as for the use of individuals who have difficulty in communicating verbally and non-verbally, and to be more specific, for the use of all individuals who cannot speak, who cannot express their emotions, for the use of normal, emergency service, newborn unit and intensive care patients, for use in the detection, interpretation and translation of health, location, neuro-status, etc. data of police, military, civil aviation, etc. personnel on active field duty, as well as integration for collecting temperature, pressure, photoplethysmography, stress, magnetic field, electromagnetic force, humidity, accelerometer, gyroscope, proximity, gas, flow, ultrasonic wave, capacitive, activity data, suitable for sensing, measuring, transmitting, interpreting and translating data.

The present invention relates to a product developed for flexible, washable surfaces that can be worn or dressed without limiting mobility, which has been put forward for use in traditional fields such as medicine and diagnostics, where surface electrodes (dry, gel, etc.) are primarily used, as well as in wearable smart platforms, smart clothing and accessories, technical and professional clothing, upholstery and coating and similar fields.

Another aim of the invention is to take measurements without causing discomfort to the wearer in measurements that require constant skin-to-skin contact, suitable for 24/7 use. It is also washable and suitable for daily use. It can be used integrated on the wearable or dressable surface without any additional processing. It has a soft surface that does not cause discomfort in skin contact in accordance with the comfort needs of the user.

A further aim of the invention is that the production method is customizable according to the choice. It can be adapted according to the need for use in more than one area. At the same time, the invention is a receiver derived from a highly resistive material with a single layer structure and the ability to collect and transmit data with multiple types of sensor compatibility.

Another aim of the invention is to form the necessary infrastructure for the adaptation of IoT systems to textiles and/or textileization thereof in accordance with textile production techniques. These production techniques include weaving, knitting, 3D knitting, 3D weaving, nanotechnology, conductive printing, nonwovens, microencapsulation, SMP and bio-textile production techniques.

Another aim of the invention is to ensure that conventional textile products and textile-based surfaces become common receptor points from which IoT systems can extract data, and the data collected from these points can be processed and individualized with artificial intelligence and more accurate data analysis can be performed.

In general terms, the aims of the invention are:
To collect high quality multi-data on the skin of an individual with a textile electrode,
To transmit the data drawn from the conductive field formed by the textile electrode to the circuit and processor that will process the data with minimum loss,
To integrate the textile electrode into wearable or dressable surfaces to meet a wide range of needs,
To integrate textile electrode and conductive pathway into loT system,
To provide classification and labeling of bodily data,
To provide signal-data verification based on previous bodily responses, data processing with artificial intelligence and machine learning to personalize the transmission,
To increase the usable process capability of loT systems with textile electrode interface.

### BRIEF DESCRIPTION OF DRAWINGS

The embodiments of the present disclosure which are summarized above and discussed in more detail below can be better understood by referring to exemplary embodiments of the present disclosure illustrated in the accompanying drawings. It should be noted, however, that the accompanying drawings only describe the typical embodiments of the present disclosure, and thus, are not to be considered as limiting the scope of the present disclosure such that other effective embodiments may also be within the scope of the present disclosure.

For ease of understanding, identical reference numerals are used where possible to indicate identical elements in the figures. Figures are not drawn to scale and can be simplified for clarity. It is contemplated that elements and features of an embodiment can be usefully incorporated into other embodiments without the need for further explanation.

FIG. 1: Whole System Technical Drawing with Main application Product, Electrode, Conductive pathway, Circuit, Software, IoT, Communication Platform

FIG. 2: ECG, GSR, EMG, EEG Data Graphs

FIG. 2A: ECG data graph

FIG. 2B: EMG data graph

FIG. 2C: GSR data graph

FIG. 2D: EEG data graph

FIG. 3: Examples of wearable products and dressable surfaces

FIG. 3A: Wearable Accessories

FIG. 3B: Dressable Surface

FIG. 3C: Wearable Product

FIG. 4: Data Transfer between Electrode, Conductive Pathway, Circuit and Software

FIG. 5: Alternative Structures of Textile Electrodes and Conductive Pathways

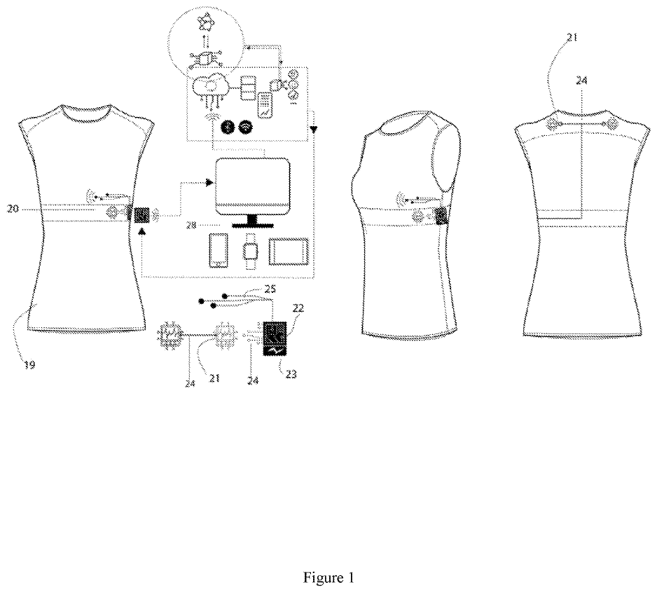

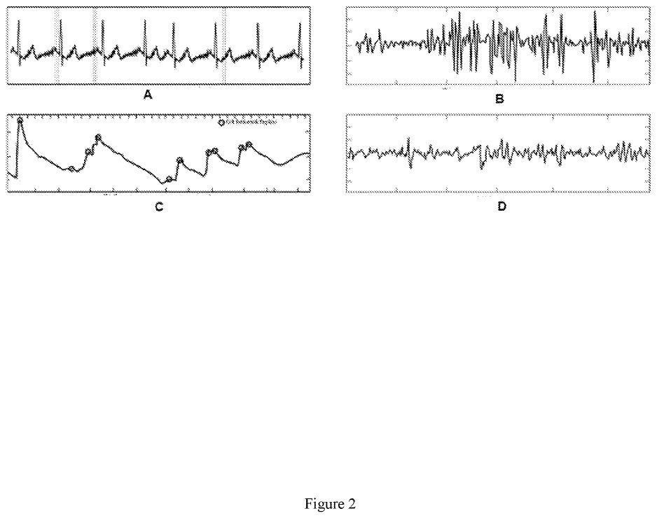

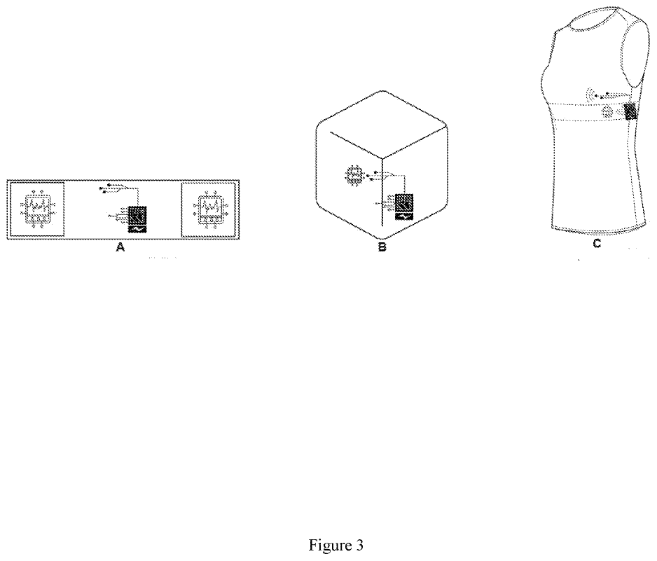

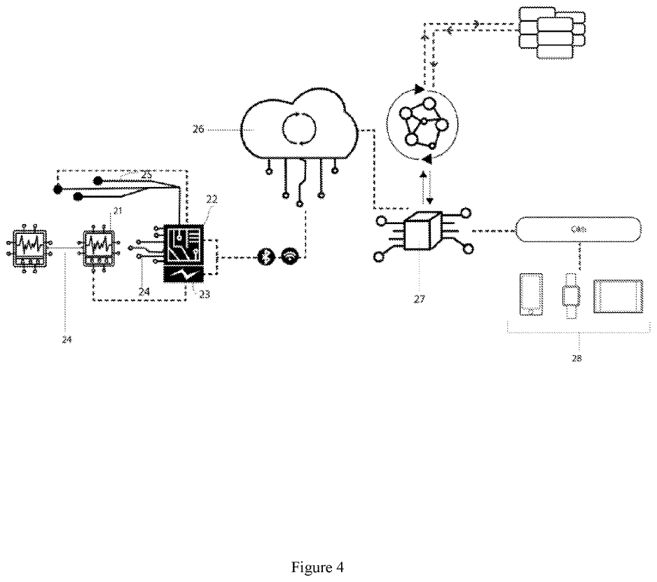

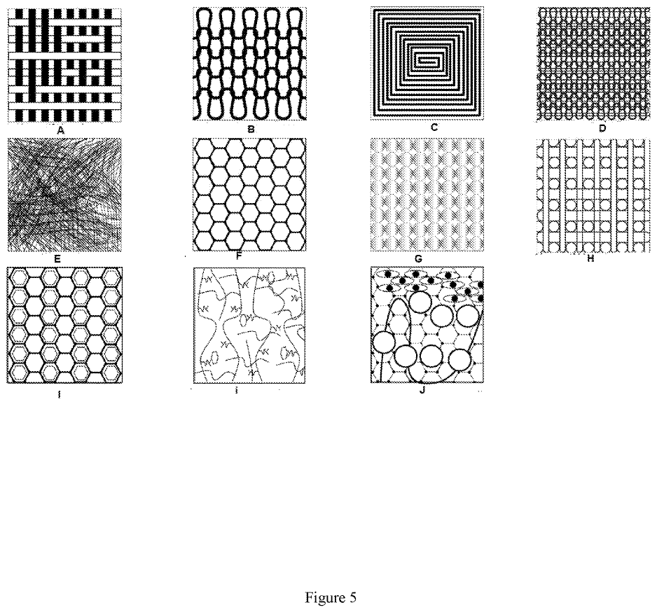

FIG-5A shows the woven structure from alternative structures of textile electrode and conductive pathway.

FIG.-5B shows the knitted structure from alternative structures of textile electrode and conductive pathway.

FIG.-5C shows the conductive printing structure from alternative structures of textile electrode and conductive pathway.

FIG.-5D shows the 3D knitted textile structure from alternative structures of textile electrode and conductive path.

FIG.-5E shows the nonwoven structure from alternative structures of textile electrode and conductive pathway.

FIG.-5F shows the nanotextile structure from the alternative structures of textile electrode and conductive pathway.

FIG.-5G shows the microencapsulation structure from alternative structures of textile electrode and conductive pathway.

FIG.-5H shows the 3D woven structure from alternative structures of textile electrode and conductive pathway.

FIG.-51 shows the 3D printing textile structure from alternative structures of textile electrode and conductive path.

FIG.-51 shows the SMP textile structure from alternative structures of textile electrode and conductive path.

FIG.-5J shows the Bio-textile structure from alternative structures of textile electrode and conductive path.

### DESCRIPTION OF DETAILS IN DRAWINGS

Described herein are the reference numbers shown in the figures.
19. Wearable/Dressable Product
20. IoT System
21. Textile Electrode
22. Circuit Board (Microchip-Microprocessor)
23. Power Source
24. Conductive Bus
25. Communication Channel
26. Cloud
27. Software
28. Response Interface

### DETAILED DESCRIPTION OF THE INVENTION

The preferred alternatives of the embodiments of invention, which are mentioned in this detailed description, are only intended for providing a better understanding of the subject-matter, and should not be construed in any restrictive sense.

The invention comprises textile electrode(s) (21), circuit board (22), power supply (23), conductive bus (24), communication channel (25), cloud (26) and software (27) in an IoT system (20) placed on a wearable/dressable product (19).

Any reaction of the body to the outside and any routine or acute vital activity that takes place within the body is reflected as an electrical signal. This signaling infrastructure is reflected on the skin as different conductivity values during response or activity. The invention enables these signals to be detected through the skin and transformed and transmitted into the data to be interpreted by the circuit to which it is connected.

In the invention, the integrated conductive material and the textile electrode (21) provide the necessary field for the detection of the instantaneous electrical conductivity difference on the skin. It combines smart textile innovations, clothing experiences and design phenomena with conventional and advanced textile manufacturing techniques to enable the transmission of data to the circuit and processor by connecting the textile electrode (21) with the sensor to be included in any smart clothing product, the electronic circuit and processor to which the sensor is connected via the conductive bus (24) proposed in the invention. The stages of formation of the textile electrode (21) and the data transmitting pathway developed with the interdisciplinary approach adopted in the invention, the creation of a data collection area with multi-sensor compatibility for the data to be collected over the skin of the individual and processed in electronic environment, taking into account the conditions of compliance with the comfort and usage conditions that the individual is accustomed to, transporting the data with minimum loss and delivering it to the circuit are followed.

The invention supports the use of wearable or dressable products and surfaces included in the smart category of individuals in continuous contact by combining data collection and transmission with wearability and ease of use, as well as supports the expansion of the usage area of smart technologies by adapting to the psychological and design product usage perceptions and expectations of individuals.

The textile electrode (21) and the data transmission pathway form a conductive field with multi-sensor compatibility, enabling the various sensors needed by the circuit for data analysis to receive data from the same field at one time. Examples of compatible on-skin data collection sensors include electrocardiography, galvanic skin response, electroencephalography and electroneuromyography. In addition, it is also suitable for integrating, sensing, measuring, transmitting, interpreting and translating data to collect temperature, pressure, photoplethysmography, stress, magnetic field, electromagnetic force, humidity, accelerometer, gyroscope, proximity, gas, flow, ultrasonic wave, capacitive, activity data within wearable products and dressable surfaces. The data received through the textile electrode (21) area and transported via the data conductive pathway can then be converted by the circuit and processor into primary level data such as heart rate, emotional sweating, muscle activation, etc., but also used to generate secondary complex data such as biobehavioral neuro-state monitoring, which involves reading, analyzing and interpreting the primary data. In daily use or in mandatory situations (hospitalization, intensive care, emergency, etc.), continuous or regular sensual contact enables the analysis to take place in any interaction that requires data analysis without disturbing the comfort and psychology of the individual and without making the individual feel the data analysis. The individual whose momentary comfort is not disturbed can easily continue their daily or essential activities. With the invention, it is aimed to perform unrestricted, uninterrupted, easy and sustainable and high quality data analysis at any condition, situation or time, and to transmit this data to the circuit with minimum loss, to personalize this data with the support of artificial intelligence with IoT system (20) integration and to adapt the feedbacks according to the user.

To describe the elements of this proposed invention in detail, it includes:
1. Textile Electrode (21): The wearable or dressable product with soft surface which is washable, suitable for daily use, adaptable in conventional or advanced textile production technologies
   a. Conductive field
   b. Non-conductive field
   c. Integration area on textile-based surface
2. Conductive Bus: The wearable or dressable product with soft surface which provides the connection between the electrode and the circuit with minimum loss, does not feel data transmission, is washable, is suitable for daily use, is adaptable in conventional or advanced textile production technologies
   a. Conductive field
   b. Non-conductive field
   C. Integration area on textile-based surface
3. Product with wearable/dressable surface
4. IoT system (20),
5. Circuit board (22),
6. Power source (23),
7. Communication channel (25),
8. Cloud (26),
9. Software (27),
10. Response interface (28).
   Briefly, the invention includes:
At least one Wearable/Dressable product that includes all IoT system (20) elements,
IOT system (20), which is the ecosystem that includes textile electrode (21), circuit board (22), power supply (23), conductive pathway, communication channel (25), cloud (26) and software (27) systems,
At least one textile electrode (21), which is a receiver that performs the functions of receiving and transmitting data so that bodily data analysis can be performed,
At least one Circuit Board (20), which is an electronic processor/card that performs the current transmission, data collection, data collection, data acquisition, data analysis and processing steps of the IoT system (22),
At least one Power Supply (23), which is the element that provides the energy flow required for the system,
At least one Conductive Bus, which is the part that connects the circuit and the textile electrode (21) parts, which performs data transmission functions so that bodily data analysis can be performed,
Communication Channel, which is the system assembly that transmits data output and communicates with other internal system/external system elements,
At least one Cloud System, which is an artificial system where the collected data is processed, stored and anonymized (26),
Software (27), which is the system that performs the processes of monitoring, detecting, analyzing sudden changes of bodily data analysis, selecting the correct label-category, classification, storage, structuring new data, updating the data scale of the system,
Response interface (28) included in the loT ecosystem, where the incoming analysis is finally transmitted to the user or the desired environment.

The steps of the method by which the invention is realized include:
Transmitting the first current from the circuit board (22) through the conductive bus (24) to the textile electrode (21),
Transmitting the incoming current to the skin by the textile electrode (21) acting as a receiver,
Returning the skin data change collected by the textile electrode (21) to the circuit board (22) via the conductive bus (24),
Detecting the skin conductivity differential difference (differential difference is only for biosensors and there are different transmission/measurement methods for detection of temperature, pressure, photoplethysmography, stress, magnetic field, electromagnetic force, humidity, accelerometer, gyroscope, proximity, gas, flow, ultrasonic wave, capacitive, activity data) with skin data change read by the software (27) embedded in the circuit board (22),
Performing the steps of reading, labeling, modeling, comparing, classifying, storing data, creating new data scales, and updating the system according to a predetermined command by the software (27),
Transfering the acquired data to the cloud (26) by circuit board (22) and embedded software (27),
Comparing newly collected data with data previously stored in the cloud (26) by software (27) embedded in the circuit board (22),
Classifying and selecting new data according to a predefined data scale and providing feedback to the circuit board (22) by software (27),
Transmitting the output by the circuit board (22) to the communication channel (25) according to the feedback received,
Transmitting the incoming feedback by the communication channel (25) to the selected response interface (28),
Performing the interface function of the entire loT system (20) by the wearable/dressable product (19),
Providing the ecosystem function including textile electrodes (21), circuit board (22), power supply (23), conductive bus (24), communication channel (25), cloud (26) and software (27) systems by the IoT system (20),
Providing the energy needed for the IoT system (20) elements by the power supply (23),
Storing the newly acquired data in the cloud (26) by software (27)
The software (27) training itself with the newly collected data scale for a personalization function to increase the accuracy of the next output response.

The wearable/dressable product (19) interface is the interface where the IoT system (20) and especially the textile electrode(s) (21) and the conductive bus (24) are integrated in order to perform processing steps such as collecting, processing, analyzing, storing, classifying, labeling, and giving feedback on the data to be measured by the user's skin contact. Textile electrode(s) (21) can be combined with, but not limited to, woven, knitted, non-woven, 3D-printed textiles, nano-textiles, microencapsulation, shape memory polymer/textile (SMP), bio-textiles, conductive printing, 3D knitting and 3D weaving techniques with conductive material integration using conventional and/or innovative textile technologies. The textile electrode(s) (21) enable the system to perform functions such as data transmission, collection, analysis, classification, labeling, storage, feedback generation, retraining of the model, etc. by transmitting the initial current sent by the circuit board (22) onto the skin and transmitting the new data generated by the conductivity differential (differential difference is only in the case of biosensors and there are different transmission/measurement methods for the detection of additional temperature, pressure, photoplethysmography, stress, magnetic field, electromagnetic force, humidity, accelerometer, gyroscope, proximity, gas, flow, ultrasonic wave, capacitive, activity data) difference in the skin back to the circuit board (22) via the conductive bus (24). Textile electrode(s) (21) are parts integrated into a washable, wearable/dressable product (19) suitable for daily use, which function as receivers to perform data processing without causing discomfort to the wearer in measurements requiring continuous skin contact. In this way, the system can continuously and constantly carry out data collection and processing steps with minimal loss without disturbing the user. Textile electrode/electrodes (21) have a uniform receiver feature that is adaptable for various and multiple sensor types such as ECG—electrocardiography, GSR—galvanic skin response, EMG—electromyography/electroneuromyography, EEG—electroencephalography, temperature, pressure, photoplethysmography, stress, magnetic field, electromagnetic force, humidity, accelerometer, gyroscope, proximity, gas, flow, ultrasonic wave, capacitive, activity, in this way, IoT system (20) measurements and bodily data analysis can be performed simultaneously in a single step without the need for different electrodes of different structures on the wearable/dressable product (19). The conductive bus (24) is responsible for data transportation by enabling communication between the textile electrode(s) (21) and the circuit board (22) and can be combined with, but not limited to, woven, knitted, non-woven, 3D-printed textiles, nano-textiles, microencapsulation, shape memory polymer/textile (SMP), bio-textiles, conductive printing, 3D knitting and 3D weaving techniques with conductive material integration using conventional and/or innovative textile technologies. The conductive bus (24) is integrated on the wearable/dressable product (19) and acts as a transmission bridge that is washable and best suited to the individual's daily wearing comfort without slipping and non-contact. The first current sent by the circuit board (22) via the conductive bus (24) to the textile electrode(s) (21) detects the differential difference (it is only for biosensors and there are different transmission/measurement methods for detection of temperature, pressure, photoplethysmography, stress, magnetic field, electromagnetic force, humidity, accelerometer, gyroscope, proximity, gas, flow, ultrasonic wave, capacitive, activity data) of the conductivity change on the skin. As a result of the first current returning to the circuit board (22), the conductor difference is analyzed by the software (27) embedded in the circuit board (22). The software (27) is a code system with cloud (26) system integration, artificial intelligence and/or machine learning features, taking the features such as desired feedback, communication channel (25) features and data quality into account. The software (27) and the cloud (26) work together, and data transfer to the cloud (26) is done via channels such as existing loT routing protocols bluetooth, WiFi, RF, BLE, cellular, LoRa, LoRaWAN, LWM2M, NFC (near field communication), Sigfox, Wi-Sun, ZigBee, Z-wave, MQTT (message queued telemetry transfer), AMQP (advanced message queue transfer), HTTP (header text transfer protocol), CoAP (Constrained Application Protocol), DDS (Data delivery service), WebSocket, XMPP (Extensible Messaging and Presence Protocol), OPC UA (OPC unified architecture). When performing functions such as data analysis, classification, labeling, responding, etc., the software (27) uses public and/or individual user data previously stored in the cloud (26) to improve the quality of analysis. The software (27) also trains and models itself with the newly acquired data to update the data scale, increase accuracy and store the new data in the cloud (26). The software (27) embedded in the circuit board (22) transmits data feedback to the board. The circuit board (22) communicates the communication solution selected according to the incoming data between the systems via the communication channel (25). These communication methods include speech channels and protocols between Internet of Things objects such as bluetooth, WiFi, RF, BLE, cellular, LoRa, LoRaWAN, LWM2M, NFC (near field communication), Sigfox, Wi-Sun, ZigBee, Z-wave, MQTT (message queue telemetry transfer), AMQP (advanced message queue transfer), HTTP (supertext transfer protocol), CoAP (Constrained Application Protocol), DDS (Data delivery service), WebSocket, XMPP (Extensible Messaging and Presence Protocol), OPC UA (OPC unified architecture). The communication channel (25) contains these speech channels and also transmits the desired feedback to the selected response interface (28). The response interface (28) includes, but is not limited to, product systems such as any loT device, computer, smart products, etc. The IoT system (20) can contain, as well as can report and give feedback on the user's physical data. A function loop is completed when the IoT system (20) transmits the last selected response by the communication channel (25) to the response interface (28). The system starts the cycle of re-measurement, analysis and response by providing the first current at any time and in cases where continuous and regular measurement is required, aiming to evaluate bodily data changes quickly and with minimal loss.

The invention is still included in the category of passive smart textiles and can be used (including but not limited to) in electronic and software systems to measure, evaluate and report data. In the next step, it is aimed to develop the invention to be included in the category of active smart textiles (Active smart textiles are textiles that can sense and report changes in the external environment) and then high smart textiles (Highly intelligent textiles are textiles that can sense and respond to changes accordingly in the external environment or adapt and change their function in response to a user input). In this way, it is planned to embed electronic components into the textile electrode (21) and transform it into a codable material, and the hardware and software will be completely realized on the invention. It is also expected to combine the material with different advanced production techniques. Techniques such as shape memory polymers, 4D printing technology (not limited) can be used that can be adapted for use in textiles in the future.

1. A flexible/washable IOT and artificial intelligence integration system suitable for daily use in the category of textile products, which can be used in on-skin data analysis that requires continuous skin contact, developed in a way that will not restrict people's mobility, the system comprising:
at least one wearable/dressable product that includes all IoT system elements;
an IOT system which is the ecosystem that includes a textile electrode, a circuit board, a power supply, a conductive data pathway, a communication channel, a cloud and software systems;
a response interface where the IoT system and especially the textile electrode(s) and the conductive bus are integrated in order to perform processing steps such as collecting, processing, analyzing, storing, classifying, labeling, and giving feedback on the data to be measured by the user's skin contact;
which is included in the IoT ecosystem, where the incoming analysis is finally transmitted to the user or the desired environment.

2. IoT system integration according to claim 1, characterized in comprising the process steps of:
at least one textile electrode that allows the initial current sent by the circuit board to be transmitted to the skin and the new data generated by the conductivity differential difference (differential difference of conductivity change is only in the case of biosensors and there are different transmission/measurement methods for the detection of additional temperature, pressure, photoplethysmography, stress, magnetic field, electromagnetic force, humidity, accelerometer, gyroscope, proximity, gas, flow, ultrasonic wave, capacitive, activity data) on the skin to be transmitted back to the circuit board via the conductive bus so that the system can perform functions such as data transmission, collection, analysis, classification, labeling, storage, feedback generation, retraining the model, in addition, which is integrated into a washable, wearable/dressable product suitable for daily use, which acts as a receiver to perform data processing without causing discomfort to the wearer in measurements requiring continuous skin contact, thus ensuring continuous and constant data collection and processing steps with minimal loss;
at least one Circuit Board which is an electronic processor/card that performs the current transmission, data collection, data collection, data acquisition, data analysis and processing steps of the IoT system;
at least one Power Supply, which is the element that provides the energy flow required for the system;
at least one Conductive Bus, which is the part that performs data transmission functions, connects the circuit and textile electrode parts and is responsible for data transport by providing communication between them in order to perform bodily data analysis;
communication channel, which is the system assembly that transmits data output and communicates with other internal system/external system elements;
at least one cloud system, which is an artificial system where the collected data is processed, stored and anonymized;
software which is the system that performs the processes of monitoring, detecting, analyzing sudden changes of bodily data analysis, selecting the correct label-category, classification, storage, structuring new data, updating the data scale of the system.

3. A method provided with textile-based on-skin data collecting electrode, conductive data transport path and IoT system integration, comprising the process steps of:
transmitting a first current from a circuit board through a conductive bus to a textile electrode;
transmitting the incoming current to the skin by the textile electrode acting as a receiver;
returning the skin data change collected by the textile electrode to the circuit board via the conductive bus;
detecting the skin conductivity differential difference (differential difference of conductivity change is only for biosensors and there are different transmission/measurement methods for detection of temperature, pressure, photoplethysmography, stress, magnetic field, electromagnetic force, humidity, accelerometer, gyroscope, proximity, gas, flow, ultrasonic wave, capacitive, activity data) with skin data change read by the software embedded in the circuit board;
performing the steps of reading, labeling, modeling, comparing, classifying, storing data, creating new data scales, and updating the system according to a predetermined command by the software;
transferring the acquired data to the cloud by circuit board and embedded software;
comparing newly collected data with data previously stored in the cloud software embedded in the circuit board;
classifying and selecting new data according to a predefined data scale and providing feedback to the circuit board by software;
transmitting the output by the circuit board to the communication channel according to the feedback received;
transmitting the incoming feedback by the communication channel to the selected response interface;
performing the interface function of the entire IoT system by the wearable/dressable product;
providing the ecosystem function including textile electrodes, circuit board, power supply, conductive bus communication channel, cloud and software systems by the IoT system;
providing the energy needed for the IoT system elements by the power supply;
storing the newly acquired data in the cloud by software;
the software training itself with the newly collected data scale for a personalization function to increase the accuracy of the next output response.

4. A textile electrode according to claim 1, includes comprising conductive field, non-conductive field and textile-based surface integration field regions.

5. The textile electrode according to claim 1, wherein it can be selected from wearable or dressable products with soft surface which provides the connection between the electrode and the circuit with minimum loss, does not feel data transmission, is washable, is suitable for daily use, is adaptable in conventional and/or advanced textile production technologies.

6. The textile electrode according to claim 1, wherein textile electrode(s) can be combined with woven and/or knitted and/or non-woven and/or 3D-printed textiles and/or nano-textiles and/or microencapsulation and/or shape memory polymer/textiles (SMP) and/or bio-textiles and/or conductive printing and/or 3D knitting and/or 3D weaving techniques with conductive material integration using conventional and/or innovative textile technologies.

7. The textile electrode according to claim 1, wherein textile electrode/electrodes have a uniform receiver feature that is adaptable for various and multiple sensor types for ECG—and/or electrocardiography, GSR—and/or galvanic skin response and/or EMG—electromyography/electroneuromyography and/or EEG—electroencephalography and/or temperature and/or pressure and/or photoplethysmography and/or stress and/or magnetic field and/or electromagnetic force and/or humidity and/or accelerometer and/or gyroscope and/or proximity and/or gas and/or flow and/or ultrasonic wave and/or capacitive and/or activity data, in this way, IoT system measurements and bodily data analysis can be performed simultaneously in a single step without the need for different electrodes of different structures on the wearable/dressable product.

8. A Conductive bus according to claim 1, wherein it can be combined with woven and/or knitted and/or non-woven and/or 3D-printed textiles and/or nano-textiles and/or microencapsulation and/or shape memory polymer/textiles (SMP) and/or bio-textiles and/or conductive printing and/or 3D knitting and/or 3D weaving techniques with conductive material integration using conventional and/or innovative textile technologies.

9. A response interface according to claim 1, wherein it includes any system of IoT devices and/or computers and/or smart products.
